(19) United States
(12) Patent Application Publication
(10) Pub. No.: US 2026/0166246 A1
(43) Pub. Date: Jun. 18, 2026

(54) INTEGRATED MANUAL VENTILATION MONITORING SYSTEM WITH REAL-TIME FLOW-BASED TIDAL VOLUME ESTIMATION AND TRAINING PLATFORM

(71) Applicant: SafeBVM Corp., Boston, MA (US)

(72) Inventor: Prathamesh Prabhudesai, Boston, MA (US)

(21) Appl. No.: 19/271,297

(22) Filed: Jul. 16, 2025

Related U.S. Application Data
(60) Provisional application No. 63/672,151, filed on Jul. 16, 2024.
(60) Provisional application No. 63/733,294, filed on Dec. 12, 2024.

(51) Int. Cl. A61M 16/00

(52) U.S. Cl. CPC A61M 16/0003; A61M 2016/003

(57) ABSTRACT

A system for reducing the risk of overventilating a subject in need of ventilation includes a ventilation assembly comprising a ventilation bag, a patient interface and an automatic flow reduction valve configured to impede flow to a subject wearing the ventilation bag when a pressure or flow rate in a flow path from the bag to the subject exceeds a maximum threshold value. A ventilation sensor positioned in or near to the flow path is configured to measure the pressure or flow rate in the flow path. The ventilation assembly is configured to communicate via a user interface or a communicably coupled device with a display at least one signal related to a maximum tidal volume determined based on signals from the ventilation sensor.

### CROSS-REFERENCES

The present application is related to U.S. Provisional Patent Application Ser. No. 63/672,151, titled “BAG VENTILATION WITH TIDAL VOLUME CONTROL,” filed on Jul. 16, 2024, and U.S. Provisional Patent Application Ser. No. 63/733,294, titled “BAG VENTILATION WITH TIDAL VOLUME CONTROL,” filed on Dec. 12, 2024, each of which is hereby incorporated by reference in its entirety for all purposes.

### FEDERALLY SPONSORED RESEARCH OR DEVELOPMENT

This invention was made with Government support under contract R44HL165932 awarded by The National Institutes of Health, under contract 2136787 awarded by The National Science Foundation, and under contract HT94252310316 awarded by The Department of Defense. The Government has certain rights in this invention.

### FIELD

The present disclosure relates generally to manual resuscitation and respiratory care, and more specifically to a system and method for measuring and predicting tidal volume in manual ventilation using integrated flow/pressure sensors. The present disclosure also relates to the use of machine learning algorithms with such systems. The present disclosure further relates to an associated training platform for improving clinical performance in manual ventilation.

### BACKGROUND

Manual resuscitation using bag-valve-mask (BVM) devices is commonly employed in emergency and critical care settings to manually ventilate patients who cannot breathe on their own, such as those in cardiac arrest, suffering respiratory distress, under anesthesia, and the like.

Conventional BVMs have a compression bag that is manually compressed by a user to pressurize and deliver a breathing gas to a patient through a breathing mask held over the patient's mouth and nose or a laryngeal mask for supraglottal ventilation or an endotracheal tube.

Poor manual technique using a BVM can be problematic. In particular, over-pressurization of the compression bag can increase both breathing gas pressure and flow rate over optimum levels, and in the worst cases can potentially harm the patient and in some cases exceed safe levels, for example causing air entry into the stomach.

A significant limitation of conventional manual ventilation systems is the absence of real-time feedback on key ventilation parameters, particularly tidal volume. Clinicians typically rely on subjective assessments and uncalibrated visual cues, which can result in over- or under-ventilation, contributing to patient harm.

Recently, the use of small ventilation bags (1000 ml) to minimize complications associated with overventilation in patients has been recommended. This recommendation has sparked considerable discussion within the medical community, especially concerning its potential impacts on patient outcomes during resuscitation.

Volume restricted bags take away providers' ability to deliver high tidal volumes and solve hyperventilation but can significantly increase risk of underventilation. For example, the delivered volume with small bags during one hand ventilation rarely crosses minimum guaranteed tidal volume (e.g., 600 ml) to compensate for mask leak. Furthermore, with use of adjuncts that increase dead space, volume restricted small bags significantly increase the risk of inadequately oxygenating the patient. Automatic pressure/flow limiting devices as described in US Patent Application Publication No. 2024/0350764 A1, which is incorporated herein by reference, help minimize high peak flows, peak pressures and volume delivered to the patient by capping the peak inspiratory flow or the differential pressure by acting as a forcing function. However, flow limiting valves don't control the absolute maximum tidal volume, a variable that is directly linked to the size of the bag valve mask. Though the flow limiting valves enable providers to deliver high tidal volume to compensate for mask leaks, in situations where providers would like feedback on maximum tidal volumes the flow limiting valve as designed is unable to do that.

Existing sensor solutions are often placed distally from the patient, and their accuracy is affected by intermediary components such as tubing, connectors, and filters. Additionally, conventional systems do not provide a forcing function combined with actionable clinical feedback or integrate data analytics to guide therapy. There exists a need for a compact, accurate, and integrated solution to monitor and predict tidal volume and flow characteristics in real-time.

Accordingly, there is a need for improved systems and methods for reducing and avoiding overventilation.

### SUMMARY

The present embodiments provide systems and methods for avoiding overventilation when using a ventilation assembly such as a BVM assembly.

Certain embodiments provide an integrated manual ventilation monitoring system including a flow sensor embedded within a resuscitation device, an electronics subsystem for signal acquisition and processing. Certain embodiments employ a machine learning model for tidal volume estimation. Certain embodiments provide a real-time visual feedback interface. In certain embodiments, an integrated manual ventilation monitoring system may be incorporated into existing manual resuscitation platforms, for example a modified version of the Sotair® manual resuscitation platform as described in US Patent Application Publication No. 2024/0350764 A1. In addition, a machine learning model may be embedded in the software associated with such existing platforms.

Certain embodiments also provide a training platform designed to improve user proficiency in manual ventilation using real-time waveform feedback and gamified training modules.

Certain embodiments disclosed herein relate to manual ventilation or manual resuscitation of a subject/patient, monitoring, provider-training, continued education, clinical research, or quality assurance and quality improvement projects (QA/QI).

According to an embodiment, a respiratory gas delivery device may include a flow-limiting device, a sensor, and a timer configured to measure an inspiratory time. Typically, the device will include a bag. The device may also include an alarm mechanism, and an alarm may be generated based on a maximum tidal value calculation. The flow-limiting device may be a gradient-type device or a binary-type device.

According to an embodiment, a system for reducing the risk of overventilating a subject in need of ventilation is provided. The system typically includes a ventilation assembly comprising a ventilation bag, a patient interface and an automatic flow reduction valve configured to impede flow to a subject wearing the ventilation bag when a pressure or flow rate in a flow path from the bag to the subject exceeds a maximum threshold value. The assembly further typically includes a ventilation sensor positioned in or near to the flow path that is configured to measure the pressure or flow rate in the flow path, a timer, and an optional user interface, the ventilation assembly configured to communicate via the user interface or a communicably coupled device with a display at least one signal related to a maximum tidal volume determined based on signals from the ventilation sensor.

According to an embodiment, the patient interface comprises a face mask or a laryngeal mask for supraglottal ventilation or an endotracheal tube.

According to an embodiment, the at least one signal related to the maximal tidal volume is determined based on one or more of an inspiratory time, an inspiratory flow time, and a peak flow rate.

According to an embodiment, the ventilation sensor detects a start and an end of an inspiratory pulse and the inspiratory flow time is measured based on the time difference between the start and the end of the inspiratory pulse.

According to an embodiment, the ventilation sensor includes one of a pressure sensor or a flow sensor located along the flow path within a device housing the automatic flow reduction valve.

According to an embodiment, the start of the inspiratory pulse is triggered by a pressure or flow rate or both above a start threshold value and the end of the inspiratory pulse is triggered by a subsequent pressure or flow reading below an end threshold value.

According to an embodiment, the flow reduction valve comprises a deformable seal having an open position and a closed position wherein the deformable seal is configured to deform to fully block the flow path in response to the pressure or flow rate exceeding the maximum threshold value.

According to an embodiment, the at least one signal related to a maximal tidal volume is a warning signal triggered when an inspiratory flow time exceeds an inspiratory flow time threshold value.

According to an embodiment, the inspiratory flow time threshold value is a maximum tidal volume determined based on an inspiratory time and flow characteristics of the automatic flow reduction valve.

According to an embodiment, the system further includes a processor configured to implement machine learning to determine the maximal tidal volume.

According to an embodiment, an assembly is provided that includes an automatic flow reduction valve with an integrated ventilation sensor and a processor The automatic flow reduction valve is configured to be mated between a ventilation bag and a patient interface and is configured to impede flow of air in a flow path from the bag to a subject in need of ventilation wearing the patient interface when the bag is compressed by a user causing a pressure or flow rate from the bag to the subject via the patient interface to exceed a maximum threshold value, and wherein the ventilation sensor is configured to detect the pressure or the flow rate of air in the flow path, and the processor is configured to measure an inspiratory flow time based on the detected pressure or flow rate at multiple time points in a ventilation cycle and to use the inspiratory flow time to generate a signal used to notify the user of a potential overventilation condition.

According to an embodiment, the flow reduction valve comprises a deformable seal having an open position and a closed position wherein the deformable seal is configured to deform to fully block the flow path in response to the pressure or flow rate exceeding the maximum threshold value.

According to an embodiment, the flow control valve may be configured to automatically and variably limit the rate of gas flow from the bag to the mask between a predetermined minimum flow rate and a maximum flow rate, instead of fully blocking the flow path.

According to an embodiment, the ventilation sensor includes one of a pressure sensor or a flow sensor located along the flow path within a device housing the automatic flow reduction valve.

According to an embodiment, a method for avoiding overventilation of a subject in need of ventilation is provided. The method typically includes (a) providing a ventilation assembly comprising a ventilation bag, a patient interface and an automatic flow reduction valve configured to impede flow to the patient interface when a pressure or flow rate in a flow path from the bag exceeds a maximum threshold value, the assembly further comprising a ventilation sensor positioned in or near to the flow path and configured to measure the pressure or flow rate in the flow path and a timer, the ventilation assembly configured to display at least one signal related to a maximum tidal volume determined based on signals from the ventilation sensor; (b) positioning the patient interface on a subject in need of ventilation; and (c) compressing the ventilation bag to deliver breathing gas to the subject and using the at least one signal related to the maximal tidal volume to avoid the overventilation of the subject.

According to an embodiment, the automatic flow reduction valve comprises a deformable seal having an open position and a closed position wherein the deformable seal is configured to deform to fully block the flow path in response to the pressure or flow rate exceeding the maximum threshold value.

According to an embodiment, the flow control valve may be configured to automatically and variably limit the rate of gas flow from the bag to the mask between a predetermined minimum flow rate and a maximum flow rate, instead of fully blocking the flow path.

According to an embodiment, the at least one signal related to the maximal tidal volume is based on one or more of an inspiratory time, an inspiratory flow time, and a peak flow rate.

According to an embodiment, the at least one signal related to a maximal tidal volume is a warning signal triggered when an inspiratory flow time exceeds an inspiratory flow time threshold value.

According to an embodiment, machine learning is used to determine the maximal tidal volume.

According to an embodiment, the ventilation sensor includes one of a pressure sensor or a flow sensor located along the flow path within a device housing the automatic flow reduction valve.

According to an embodiment, a method for avoiding overventilation of a subject in need of ventilation is provided. The method typically includes providing a ventilation assembly comprising a ventilation bag, a patient interface and an automatic flow reduction valve configured to impede flow to the subject when a pressure or flow rate from the bag exceeds a maximum threshold value. The assembly further includes a ventilation sensor (e.g., ventilation waveform sensor such as a pressure sensor or flow sensor to provide pressure and/or flow rate signals) and a timer. The ventilation assembly is configured to display, e.g., on a monitor or display device, at least one signal related to a maximum tidal volume determined using the ventilation waveform sensor. The method further typically includes compressing the ventilation bag to deliver breathing gas to the subject while using the at least one signal related to the maximal tidal volume to avoid the overventilation of the subject.

According to an embodiment, the patient interface includes a face mask or a laryngeal mask for supraglottal ventilation or an endotracheal tube.

According to an embodiment, the signal related to the maximal tidal volume is an inspiratory time (IT) signal.

According to an embodiment, the signal related to the maximal tidal volume is determined based on one or more of an inspiratory time, an inspiratory flow time or a peak flow rate. The peak flow rate is a rate that may be equal to or less than the maximum flow rate allowed by the flow reduction valve.

According to an embodiment, the ventilation sensor detects a start and an end of an inspiratory pulse and an inspiratory time is measured based on the time difference between the start and end of the inspiratory pulse.

According to an embodiment, the start and end of the inspiratory pulse are detected by a pressure or flow sensor located between the bag and the patient interface.

According to an embodiment, the pressure or flow sensor is located in or in proximity of the automatic flow reduction valve.

According to an embodiment, the start of the inspiratory pulse is triggered by a pressure or flow or combination of both, with or without a logic reading above a start threshold value and the end of the inspiratory pulse is triggered by a subsequent pressure or flow reading combination of both readings below an end threshold value.

According to an embodiment, the flow reduction valve includes a deformable seal having an open position and a closed position wherein the deformable seal is configured to deform to fully block the flow path in response to the pressure or flow rate exceeding the maximum threshold value.

According to an embodiment, the at least one signal related to a maximal tidal volume is a warning signal triggered when an inspiratory time exceeds an inspiratory time threshold value.

According to an embodiment, the flow control valve may be configured to automatically and variably limit the rate of gas flow from the bag to the mask between a predetermined minimum flow rate and a maximum flow rate, instead of fully blocking the flow path

According to an embodiment, the inspiratory threshold value is a maximum tidal volume determined from the inspiratory time and the flow characteristics of the automatic flow reduction valve.

According to an embodiment, the sensor(s) connect to a monitor and provide visual, auditory or tactile cues to the user to modify ventilation technique through the monitor or another similar platform that implements the signal processing and/or signal or data display.

According to an embodiment, a large adult bag may advantageously be converted into a functional small adult bag by controlling inspiratory time with a flow limiting device to cap a maximum delivered tidal volume. This large adult bag addresses the weaknesses of the small adult bag where the small adult bag is a forcing function and cannot allow volumes larger than its capacity. Whereas the flow limiting valve with maximum delivered tidal volume control, is not a forcing function for volume, but just a feedback on tidal volume while acting as a forcing function for peak flow rate.

In certain aspects, the type of flow limiting device and the flow limiting threshold may vary. For example, a fully block flow limiting device would essentially result in a change in behavior of the provider and the provider will maintain standard manual ventilation waveforms at a flow rate lower than that of the threshold. In this case, a higher flow threshold would result in higher maximum tidal volume and a lower threshold will result in a lower maximum tidal volume for the same inspiratory time.

Similarly, for a constant flow rate (air leak) at a flow limiting threshold type of device, for the same inspiratory time, the device with a higher threshold will result in a higher max volume than a device with a lower threshold.

Comparison between the types of flow limiting devices may depend on the human factors component of how the provider delivers air with an air leak type of flow limiting threshold. The provider could immediately stop or the provider could continue delivering air with a constant leak flow. In the case of the latter, if both types of limiting devices have the same threshold, then the leak type will result in a higher tidal volume than a fully blocking device as the leak type will have a larger area under the curve and waveforms with plateaus.

In a further embodiment, a non-transitory computer readable medium is provided that stores instructions, which when executed by one or more processors, cause the one or more processors to control a ventilation assembly (e.g., integrated manual ventilation monitoring system including an assembly having an automatic flow reduction valve and a flow sensor) to implement any of the methods as described herein. The one or more processors may be integrated in one or more components of the ventilation assembly.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE SEVERAL VIEWS OF THE DRAWINGS

The detailed description is described with reference to the accompanying figures. The use of the same reference numbers in different instances in the description and the figures may indicate similar or identical items.

FIG. 1A shows a perspective view of an automatic flow reduction valve assembly according to an embodiment.

FIG. 1B shows a cross-sectional view of the automatic flow reduction valve assembly of FIG. 1A.

FIG. 2 shows an example embodiment of a process of ventilating a patient according to embodiments herein.

FIG. 3A, FIG. 3B and FIG. 3C illustrate example display outputs that includes pressure and flow measurements and values derived therefrom, including inspiratory flow time, using an integrated manual ventilation monitoring system according to embodiments.

FIG. 4 shows scatter plots of Peak Inspiratory Pressures (PIP) for breaths delivered by 217 providers with and without the Sotair pressure safety device.

FIG. 5 shows scatter plots of Tidal Volume for breaths delivered by 217 providers with and without the Sotair pressure safety device.

FIG. 6, shows a breath detection algorithm leveraging a state machine comprising three states: Positive, Zero, and Negative, according to an embodiment.

FIG. 7 shows a clinical trial software module to address imperfections in the data, wherein the software module may include various sub-modules, according to an embodiment.

FIG. 8 shows an example of a real-time data recording displayed on a main window on a user interface according to an embodiment.

FIG. 9 shows an example of a data viewer and data validation window displayed on the user interface according to an embodiment.

FIG. 10A, FIG. 10B and FIG. 10C show examples displays of Inspiratory Flow time Feedback and Flow Limiting Valve Activation according to embodiments.

FIG. 11A and FIG. 11B show examples of flutter for breath flows.

FIG. 12 shows that inspiratory time and peak flow time contribute in nearly equal measure to the estimate of tidal volume.

FIG. 13 shows an example process flow according to an embodiment.

FIG. 14, FIG. 15 and FIG. 16 illustrate examples of user interface displays embodying the interactive gamified environment according to an embodiment.

### DETAILED DESCRIPTION

Presently disclosed embodiments provide systems and methods for avoiding overventilation when using a ventilation assembly such as a BVM assembly. Certain embodiments provide an integrated manual ventilation monitoring system including an assembly having an automatic flow reduction valve, a sensor embedded within a flow path of a resuscitation device, and an electronics subsystem for signal acquisition and processing. In some embodiments, a model for tidal volume estimation, and/or a real-time visual feedback interface are provided. Certain embodiments also provide a training platform designed to improve user proficiency in manual ventilation using real-time waveform feedback, optionally with gamified training modules.

According to various embodiments, a ventilation system includes a ventilation interface (a positive pressure source of breathing gas and an airway) such as a Bag-Valve-Mask (BVM), which can further comprise an automatic flow reduction valve (may also be referred to as “flow limiting valve” or “flow control valve”). The valve operates to impede flow of a breathing gas to a subject (e.g., a patient in need of resuscitation) when the rate of flow of breathing gas from a source (e.g., a BVM bag) exceeds a threshold value. The ventilation assembly further comprises a ventilation waveform sensor (e.g., pressure sensor or flow sensor) and a timer and may be configured to display at least one signal related to the maximum tidal volume and/or activation of the flow limiting valve, (e.g., a measured inspiratory flow time) as determined by the ventilation waveform sensor (hereinafter also referred to as “ventilation sensor”). The display can be, for example, provided in a component directly coupled with the sensor, or on a monitor or other display device remote from the sensor. For example, the ventilation sensor may detect the temporal length of an inspiratory pulse delivered to the subject by the user/caregiver and a display of the inspiratory time or inspiratory flow time or other relevant information may be displayed to the user. In this context, the inspiratory flow time is the inspiratory time minus the time that flow is paused. The measurement and display can be repeated multiple times per respiratory cycle and over multiple respiratory cycles. Optionally, the assembly can provide guidance to the user as to the appropriateness of the length of the inspirations and/or appropriate use of a manual resuscitator with a flow limiting valve. Such guidance can take various forms, such as a displayed icon or color, a measurement, a score, or an alarm. The guidance can provide immediate feedback to the caregiver, or a measure used for retrospective QA/QI or group training.

In an embodiment, an inspiratory flow volume or maximal tidal volume is calculated based on the assumption or knowledge that the inspiratory flow rate is governed by the maximum for of a flow limiting valve.

Alternately or in addition, an inspiratory flow volume can be determined from the peak flow volume of an inspiratory waveform. This method can be applied when the flow is less than the maximum allowed by a flow limiting valve or when a flow limiting valve is not present.

### Device/System

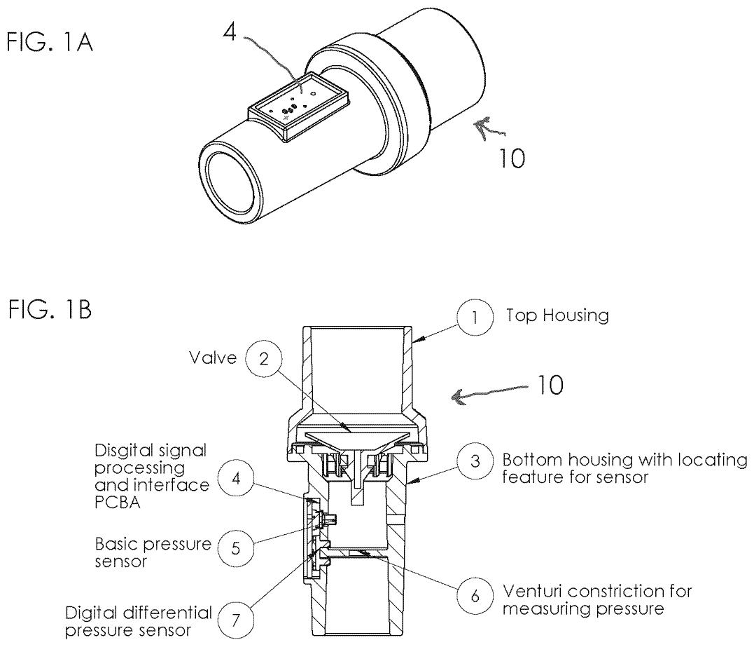

FIG. 1A and FIG. 1B show an embodiment of an automatic flow reduction valve assembly 10. Assembly 10 includes a top housing 1 and a bottom housing 3. Top housing 1 includes an opening or port configured to be connected to a port of a ventilation bag (e.g., configured as a bag port), or to an intermediary device that may connect to a ventilation bag, and a bottom housing 3 includes an opening or port configured to be connected to a patient interface such as a patient mask (e.g., port on bottom housing may be connected to a mask connector on a patient mask) or to an intermediary device that may connect to a patient interface. A flow path is defined within assembly 10 from the bag port of top housing 1 to the mask fitting of bottom housing 3. For example, assembly 10 may be advantageously used with a bag valve mask (BVM) for preventing over-pressurization. A BVM typically includes a bag assembly having a bag connector for detachably mating to a mask connector on a patient mask and the assembly 10 may be connected therebetween; the bag port of assembly 10 may detachably connect to the bag connector on the BVM, and the mask fitting of assembly 10 may detachably connect to the mask connector on the BVM. It is noted that “bottom” and “top” are used herein solely with reference to FIG. 1B, and that there is no actual orientation requirement of the housing structure of valve assembly 10.

Assembly 10 also includes an automatic flow reduction valve 2, which may be a binary-type flow-limiting valve or gradient type valve or other type of valve, and at least one ventilation sensor 5, 6 and/or 7 located in or near the flow path within the bottom housing 3 as shown in FIG. 1B. Automatic flow reduction valve 2 operates to impede flow when pressure on a bag connector side of the valve exceeds a maximum threshold value. For example, the automatic flow reduction valve may include a deformable seal having an open position and a closed position and the deformable seal is configured to deform to fully block the flow path in response the pressure or flow rate exceeding the maximum threshold value. The deformable seal may be in an unstressed configuration in the open position and in a stressed configuration in the closed position. Additionally, the deformable seal may have a conical periphery configured to evert from the unstressed configuration to the stressed configuration in response the pressure or flow rate exceeding the maximum threshold value. Reference is made to U.S. Pat. No. 12,017,008 B2, which is incorporated by reference herein, for structural and operational features of a pressure safety device including an automatic flow reduction valve.

Ventilation sensor 5, 6 and/or 7 may include a pressure or flow sensor, such as a digital mass flow sensor, integrated directly into assembly 10. The sensor is located proximally to the patient (e.g., proximally to patient interface) along the flow path within bottom housing 3 to minimize dead space and measurement error. The sensor 5, 6 and/or 7 is configured to measure either or both of inspiratory and expiratory flow parameters and may output analog or digital data via a calibrated and temperature-compensated signal pathway. Onboard electronics subsystem 4 is provided to interface with sensor 5, 6 and/or 7, and provide signal processing, communications (e.g., wired or wireless communications with remote devices) and/or readout (e.g., display) functionality on assembly 10. Electronics subsystem 4 includes a timer (not shown) to assist with various measurements as described herein.

The onboard electronics subsystem 4 include a processor such as a microcontroller and may include a communications interface configured to send the digitized flow data to one or more of a local embedded processor for on-device calculation and visualization (e.g., via a display; subsystem 4 may include an embedded display device), a connected external device (e.g., computer, tablet) utilizing software such as Sotair IQ Clincal, or a cloud-based analytics platform for post-processing such as Sotair IQ Clinical Cloud.

Examples of patent interfaces, ventilation bags, ventilation/flow sensors, processors, timers and other components useful in various embodiments herein will be described in more detail below.

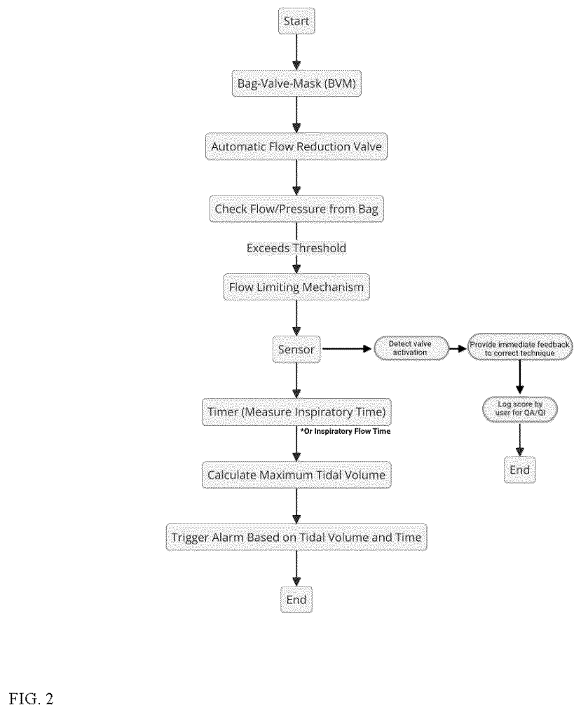

An embodiment includes a method of using the assembly to ventilate a patient, provide feedback and/or alarms and determine various system and flow parameters is shown in FIG. 2. An advantage of the embodiments herein is that the patient can be more safely ventilated with lower risk of volutrauma or barotrauma. FIG. 2 shows an example embodiment of a process of ventilating a patient using an integrated manual ventilation monitoring system including an assembly 10 according to embodiments herein. For example, the method may generally include (a) providing a ventilation assembly comprising a ventilation bag, a patient interface, a ventilation sensor positioned in or near to the flow path and an automatic flow reduction valve configured to impede flow to the patient interface when a pressure or flow rate in the flow path from the bag exceeds a maximum threshold value; (b) positioning the patient interface on a subject in need of ventilation; and (c) compressing the ventilation bag to deliver breathing gas to the subject and using the at least one signal related to the maximal tidal volume to avoid the overventilation of the subject. The assembly may further be configured to display at least one signal related to a maximum tidal volume determined based on signals from the ventilation sensor.

In operation, with the automatic flow reduction valve assembly 10 mated between a ventilation bag and a patient interface, valve 2 is configured to impede flow of air in the flow path from the connected bag to a subject in need of ventilation wearing the patient interface when the bag is compressed by a user causing a pressure or flow rate from the bag to the subject via the patient interface to exceed a maximum threshold value, and the ventilation sensor 5, 6 and/or 7 is configured to detect the pressure or the flow rate of air in the flow path, and the onboard electronics subsystem 4 is configured to measure an inspiratory time or and inspiratory flow time based on the detected pressure or flow rate at multiple time points in a ventilation cycle and to use the inspiratory flow time to calculate various parameters including a maximal tidal volume as well as to generate a signal (e.g., an alarm) used to notify the user of a potential overventilation condition, e.g., via display on subsystem 4 or other display.

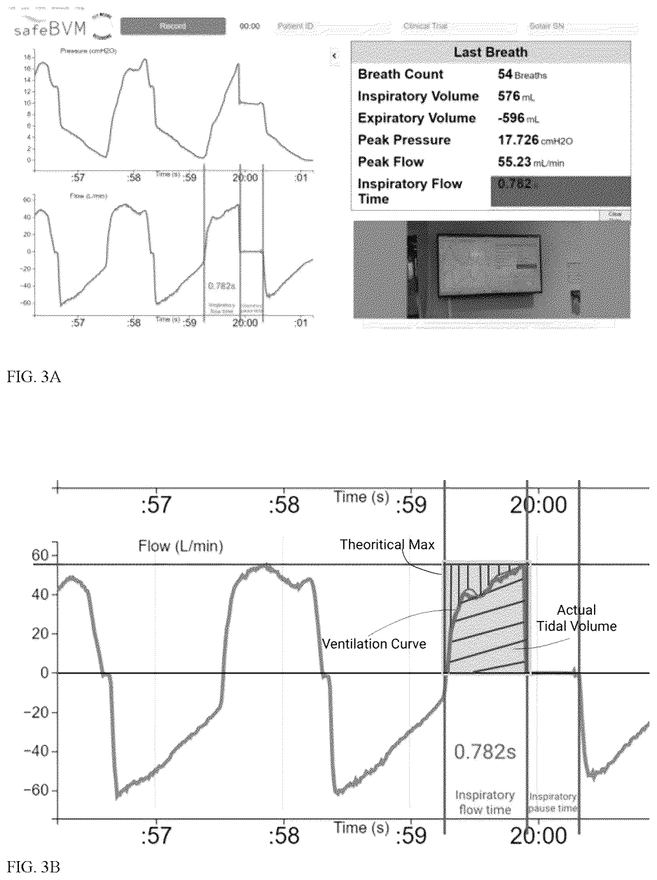

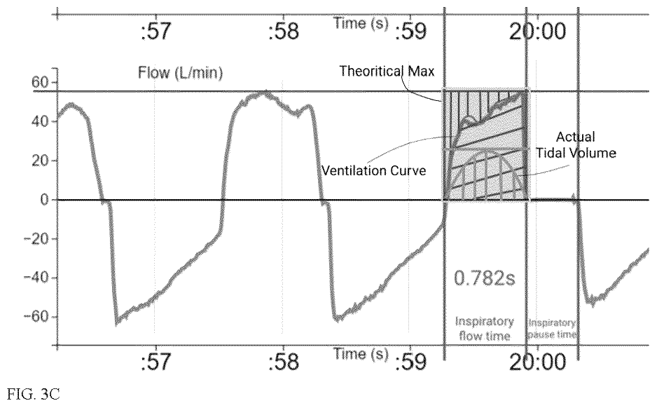

FIG. 3A illustrates an example display output that includes pressure and flow measurements and values derived therefrom, including inspiratory flow time, according to an embodiment using an integrated manual ventilation monitoring system including an assembly 10. In the zoomed-in view of FIG. 3B, a binary-type flow-limiting valve activates at a threshold flow rate value, e.g., 55 LPM, represented by a blue horizontal line parallel to the time (x-axis). Tidal volume, calculated as the integral of flow over time, is constrained by the fixed upper flow threshold. Consequently, the inspiratory flow duration becomes the primary determinant of the tidal volume. The theoretical maximum tidal volume is depicted as the gray-shaded area enclosed within the yellow boundary. The actual tidal volume is represented by the highlighted area, as labeled.

The pink curve (demarking the horizontal hash lines and the vertical hash lines) illustrates the waveform generated by a provider using a standard manual resuscitator bag. The specific characteristics of this waveform, including its shape, were analyzed to predict the maximum tidal volume. These predictions are based on the study of inspiratory times and associated tidal volumes derived from a multitude, e.g., over 40,000, of recorded breaths using flow-limiting valves. Note that the waveform and tidal volume will vary with the type of bag used.

Predicting maximum delivered tidal volume may be performed as follows:
Use flow limiting device and only measure time variable on the X axis (may provide less accuracy, can be integrated into other devices);
Use flow limiting device and measure time variable on the X axis and Maximum flow variable on the Y axis (provides more accuracy, and doesn't need continuous measurement like current standard); or
use prediction algorithms to predict max tidal volumes. These algorithms will be positive pressure ventilation device agnostic, but will become more accurate if fine-tuned for each positive pressure ventilation device. (The pink line from FIG. 3B is specific to the bag.)

Theoretical Max with the flow limiting device—Tidal volume is the area bound by the yellow box as shown in FIG. 3B.

Theoretical Max with the flow limiting device (only time measured)—Actual Max Tidal volume takes into account the pink line and the area under the ventilation curve. This pink line is predictive of the bag and set flow limiting threshold of the flow limiting device.

Theoretical Max with the flow limiting device (Peak Flow and Time measured)—As shown in FIG. 3C, the Actual Max Tidal volume takes into account the measured peak flow with the green line and the area under the green ventilation curve. This green curve is predictive of the volume using the peak flow for that breath and the measured time. Similar to the pink curve, the green curve is predictive of the positive pressure device but can also be bag agnostic. To elaborate further on this, an example is provided. The capacity of a standard bag (positive pressure device) is 1.6 L. This is the max tidal volume for any breath delivered with this ventilation device. Max Volume 1.6 L

When a flow limiting device (regardless of flow limiting threshold) is included, the provider can still squeeze the bag over a prolonged inspiratory time and deliver 1.6 L. Max Volume 1.6 L

Now with a flow limiting device that limits the flow to 55 LPM (blue horizontal line), and the time (inspiratory flow time/inspiratory time) capped for example to 1 second, the max volume is limited to 916 ml.

But depending on the positive pressure device, especially with a manual device, it is challenging to maintain 55 LPM for the entire inspiratory cycle for 1 second. Most common types of bag valve manual resuscitator devices have a pink ventilation curve and one can predict the max tidal volume for this ventilation device. At 55 LPM flow limitation for a 1 second inspiratory time, this pink ventilation line results in a max predicted volume between 500-750 ml.

Alternately or in addition, one can add the ability to predict peak flow rate, and add it as a variable to this calculation, with or without a flow limiting device. The max predicted volume will be lower than 500 ml as the peak flow rate is shifted for that breath from 55 LPM (blue horizontal line) to green horizontal line. And the function green ventilation curve will be close to the pink curve in terms of equation as it is correlated to the positive pressure ventilation device.

Thus, using an appropriate model or function with inputs of peak flow and inspiratory time, one can advantageously estimate actual tidal volume. Notably, this approach allows estimation of actual tidal volume for delivered inspirations where the flow is below the maximum flow rate enforced by a flow limiting device. An advantage of this method is that a less expensive or resource intensive sensor may be employed. For example, the sensor may not need to take numerous flow or pressure measurements over time but merely record the peak flow and the temporal bounds of the inspiration. The technique is also useful for estimating actual tidal volume for devices that lack a flow-limiting valve.

An example of a gauge for measuring peak expiratory flow rate (PEFR), e.g., maximum airflow during a forced exhale, typically measured in L/min or L/s. and time per breath (e.g., timing parameters such as time to reach peak flow (“rise time”) and total exhalation duration) within each exhalation is the Philips Respironics PersonalBest Full Range Peak Flow Meter. Other sensors that may be used to measure PEFR and time per breath include:

### Mechanical Impeller+Hall-Effect Sensor

A small impeller (mini turbine) mounted inside airflow path.
A magnet on the impeller; placed so that each rotation changes the magnetic field.
Hall-effect sensor detects this magnetic change and outputs pulses.
Pulse frequency correlates with instantaneous flow rate; time between pulses yields timing data.
Proven, cost-efficient method; components are inexpensive and widely used.
Example: Texas Instruments mechanical flow meter eval kit.
   Pneumotachograph with Differential-Pressure Sensor
Uses a fixed flow constriction and measures the pressure drop across it.
Output voltage proportional to flow rate; integrated over time gives volume and breath duration.
Very accurate but requires differential-pressure transducer and firmware.
Slightly higher cost and complexity than mechanical impeller.

### MEMS/Thermal or Ultrasonic Sensors

MEMS thermal sensors use micro-heaters with upstream/downstream sensors.
Ultrasonic sensors measure transit time differences to determine flow rate.
High accuracy, no moving parts, but components tend to be more expensive.
A similar mechanism can be used to measure Peak Inspiratory Flow Rate (PIFR).

### Ventilation Bags, Flow Limiting Devices

Bag Valve Masks (BVMs) are hand-held devices used to provide positive pressure ventilation to patients who are not breathing or are breathing inadequately. They are essential tools in emergency and critical care settings, allowing caregivers to deliver breaths to a patient by squeezing the bag, which pushes breathing gas (e.g. air or oxygen) into the lungs. BVMs often include an oxygen reservoir and can be connected to an oxygen source to deliver higher concentrations of oxygen. They are used in various situations, from routine medical emergencies to advanced life support.

While other mechanisms to deliver the breathing gas could be used, the most common mechanism in use is a ventilation bag. Typically, the bags are elastic and, due to their elastic nature, will refill with breathing gas automatically upon release of compression force by the caregiver. Being manual devices, there is a lot of variability in how providers deliver manual ventilation with the breathing bags. This variability often results in complications like gastric insufflation, aspiration pneumonia, hypoxia, high intrathoracic pressure causing lower preload, hypocapnia, hypotension, barotrauma, that reduce neurologically intact survival rates for patients. These variables can be controlled by controlling peak pressures, tidal volumes, and minute ventilation (a product of rate and volume over a minute).

Flow limiting devices address variability in peak flow rate. Peak flowrate has 95%+ correlation with peak pressures. Thus, flow limiting devices described in US20240350764A1 and U.S. Pat. No. 6,792,947B1, both commercially available as FDA cleared devices are intended for minimizing complications with peak pressures i.e. gastric insufflation (K212905) and (K021328). In particular US20240350764A1 describes a fully block flow limiting device i.e. a binary mechanism of action, on the contrary U.S. Pat. No. 6,792,947B describes a gradient/leak type mechanism where the device doesn't fully block and permits flow; it variably limits the rate of gas flow from the bag to the mask between a predetermined minimum flow rate and a maximum flow rate.

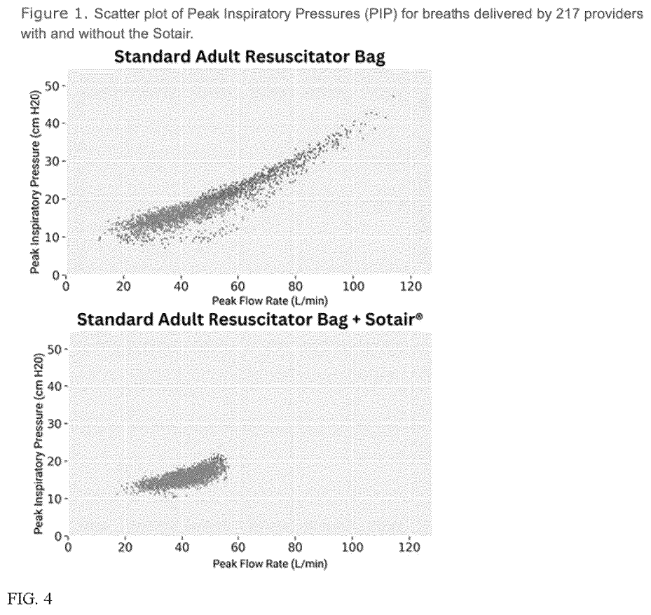

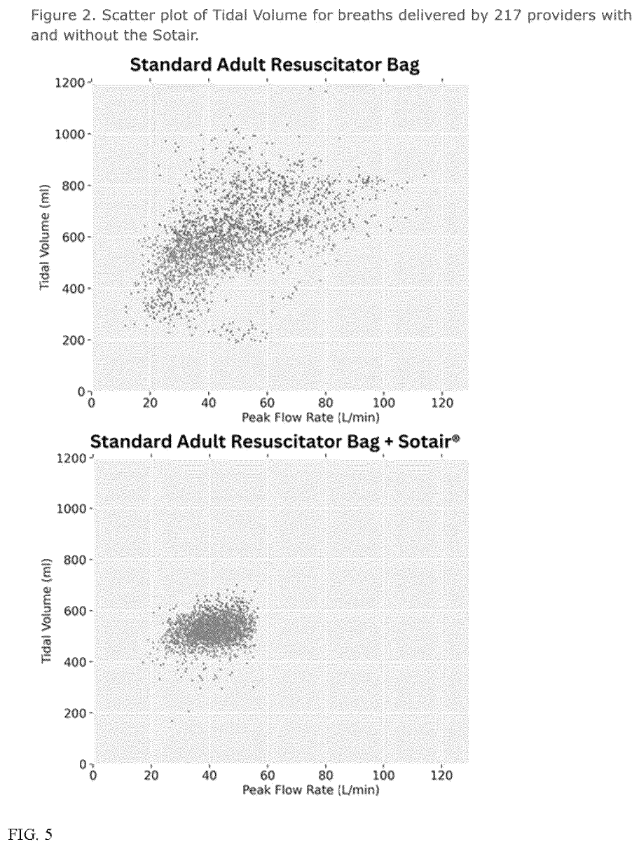

Both flow limiting devices indirectly control other parameters of manual ventilation that can be associated with complications of manual ventilation. For example, U.S. Pat. No. 6,792,947B—device described in K021328—Demonstrated effectiveness in preventing hypocapnia, as reported by Lovat et al. (Lovat R, Watremez C, Van Dyck M, et al. Smart Bag vs. Standard Bag in the Temporary Substitution of Mechanical Ventilation. Intensive Care Med. 2008; 34(2):355-360. doi:10.1007/s00134-007-0850-5). And US20240350764A1—device described in K212905—demonstrated significant control on peak pressure and tidal volumes compared to traditional BVMs, as reported by Kumar et al. (Kumar, P; Holley, J; Justice, J; Slutsky, A; Brady, M. “Manual Ventilation Performance in First Responders using a Flow-Rate Limiting Device (Sotair)” poster presentation at national American Academy of Emergency Medicine (AAEM25) annual conference, 2025.) and as shown in FIG. 4 and FIG. 5.

Flow rate being Volume/Time, it is natural for both types of flow limiting devices to impact variables related to volume. However, if a second variable like time is controlled the functionality of all flow limiting devices can be significantly improved and more precisely controlled.

The method of breath delivery typically includes compressing the ventilation bag to deliver breathing gas to the subject while using at least one signal related to the maximal tidal volume to avoid the overventilation of the subject.

Example of commercially available ventilation bags that can be used in connection with embodiments herein include:
Ambu Spur II
Curaplex Resuscitation Bags
Curaplex VentiSure 2
AirLife Adult Manual Resuscitator
Sunmed/Ventlab AirFlow Standard
Mercury Medical CPR 2, Adult CPR-2 Bag
1st Response Adult Manual Resuscitator with Oxygen Reservoir Bag
The BAG II Resuscitator Adult w/Mask #5
Adult BagEasy Resuscitator w/Mask
Disposable Bag Mask Resuscitator
RUSCH Manual Pulmonary Resuscitator with Oxygen Reservoir Bag
SmartBag MO

### Patient Interface

The patient interface/airway may include, for example a face mask, supraglottal airway, which can be a laryngeal mask, or an endotracheal tube, oropharyngeal airway, or tracheostomy tubes.

Other than using a ventilation mask applied to the face, an alternative method of gas delivery is using a supraglottic airway. Supraglottic devices, such as the I-gel and Laryngeal Mask Airway (LMA), are useful tools in airway management, particularly in situations requiring rapid and secure airway access. For example, the I-gel, a second-generation supraglottic device, is known for its non-inflatable cuff that conforms anatomically to the patient's airway, providing a reliable seal and minimizing trauma during insertion. The LMA, one of the first supraglottic airway devices introduced, features an inflatable cuff that sits above the glottis, creating a seal around the laryngeal inlet. Both devices are widely used in anesthesia and emergency medicine due to their ease of use, reduced risk of aspiration, and effectiveness in maintaining an open airway during surgery or in critical care settings.

However, supraglottic Airway Devices operate based on seal pressures. When a breath is delivered, if a pressure crosses the 20-25 cmH₂O level, leaks can occur. A flow limiting valve device such as Sotair(®), from SAFEBVM Inc., reduces pressures, ensuring adequate seals on the supraglottic airway device. High pressures in the system can also be caused due to excessive tidal volumes. These high pressures are especially aggravated when the compliance of the lung is lower, like in disease states like collapsed lungs, pneumothorax, ARDS, lung fibrosis, etc. Flow limiting devices are known to reduce high peak pressures associated with high peak flow rates. However, when flow limiting devices are used without limitation on estimated max tidal volumes variables and its dependent like inspiratory flow time or inspiratory time, their efficacy can be reduced due to high plateau pressures resulting from high tidal volumes. In these cases, the known efficacy of flow limiting devices in improving safety and effectiveness of supraglottic airways is not reaching its full potential. However, embodiments disclosed herein provide for regulating/guiding providers on inspiratory flow time and can effectively train providers to control the high tidal volumes with flow limiting devices, thereby improving the overall functionality of the system comprising of flow-limiting devices, inspiratory flow time feedback/max tidal volume control and supraglottic airway system.

In an embodiment, a visual cue such as a flashing light that glows for the inspiratory flow time threshold and blinks every six second can be used to guide provider. These blinking lights are commonplace in some ventilation equipment, and primarily used to govern the rate. Bagging every six second results in a standard respiratory rate of 10 breaths per minute. Utilizing this same light as a platform, one can guide the user on inspiratory flow time in addition to the rate at which provider bags.

### Integrated Sensor

In an embodiment, a sensor subsystem includes one or more pressure and/or flow sensors (e.g., sensors 5, 6, 7) that detect the start and end of the inspiratory pulse to measure inspiratory time. These sensors may include or be coupled with electronics configured to communicate with a display device or monitor or separate system including a monitor or display device, providing the user with real-time visual and auditory cues as the appropriateness of the inspiratory pulses in terms of predicted tidal volume. By processing signals related to maximal tidal volume and inspiratory time, the sensor subsystem can help to avoid overventilation and ensure the delivery of more precise ventilation.

Different types of flow and pressure sensors that may be used in various embodiments include: differential pressure sensors, strain-gauge (e.g., in which foil or silicone strain gauges are arranged as a Wheatstone bridge), capacitance pressure transducers, piezoelectric pressure sensors as well as MEMS pressure sensors (e.g., based on materials the change resistance with shape or force, where voltage relates to resistance and pressure) thermistor pressure sensors, based on resistance and thermal impedance, and other sensor types.

### Differential Pressure Sensors

Differential pressure sensors measure the difference in pressure between two points. In medical applications, these points might be on either side of a flow element (such as a tube or a chamber) through which a fluid (like air or gas) passes.

A differential typically will include pressure ports (two ports that connect to the locations where pressure is to be measured) and a sensing element (sensing elements can be capacitive, piezoresistive, or strain gauge elements). When a fluid flows through the device being measured, it creates a pressure differential across the flow path. The pressure differential is then converted into an electrical signal proportional to the pressure difference, via a piezoresistive/strain gauge element (Wheatstone bridges are very common in this case).

### CMOSens Technology—Complementary Metal-Oxide-Semiconductor

### Sensirion® Sensors

Sensirion's flow sensors are very high precision to accurately measure airflow and respiratory parameters in both medical and industrial applications. Sensirion sensors have multiple applications: measuring differential pressure as well as flow, either as mass flow or volume flow. Sensirion's sensor works on CMOSens® which is an evolution of hot-wire anemometry.

As outlined in the Techbriefs article “When you need a machine for breathing you′d better have accurate sensors” from Jun. 1, 2019, by Dr. Andreas Alt, Sales Director Medical, Sensirion AG, the Sensirion hot wire anemometer uses MEMs technology for flow sensors, with an on-chip micro-heater: temperature sensors placed before and after the heater. At zero flow, the temperature is the same. When flow occurs, the temperature difference between them varies depending on the amount, direction, and gas. The sensor is calibrated to operate well in low-flow/zero-flow and delivers precise readings without needing offsets.

Hot-wire anemometers have set the standard for airflow measurements in respiratory applications for decades. In these, a thin wire is placed in the gas stream and heated. By measuring the heat loss of the wire, the velocity or flow of the stream can be determined. Such hot-wire anemometers are typically analog devices—sensitive to shock and vibration—that age over time and require frequent re-calibration. Sensirion's CMOSens Technology is the natural evolution of hot-wire anemometry. It uses the same thermal measurement principle to determine the flow of a gas or liquid, but the sensing element has been miniaturized and combined with analog and digital signal processing circuitry on a tiny CMOS silicon chip. The result is an accurate, robust, cost-effective, digital, and fully calibrated solution that has been widely accepted across the industry. In the medical field, the CMOSens® Technology may be used in respiratory applications such as ventilators, anesthesia, or continuous positive airway pressure (CPAP) devices.

Sensirion sensors can be used in various measurement configurations including differential pressure measurements (dp between two chambers), flow measurements: mass flow (standard volume flow), or volume flow.

Sensirion's “Differential Pressure Sensor Selection Guide” also offers guidance on temperature compensations, in cases in which it is required (usually for volume flow) and pressure compensations (when measuring differential pressures).

### Microcontroller/Processor

The term “processor” or alternatively “controller” is used herein generally to describe various apparatus relating to the operation of one or more firmware. A controller can be implemented in numerous ways (e.g., such as with dedicated hardware) to perform various functions discussed herein. A “processor” is one example of a controller which employs one or more microprocessors that may be programmed using software (e.g., microcode) to perform various functions discussed herein. A controller may be implemented with or without employing a processor and also may be implemented as a combination of dedicated hardware to perform some functions and a processor (e.g., one or more programmed microprocessors and associated circuitry) to perform other functions. Examples of controller components that may be employed in various embodiments of the present disclosure include, but are not limited to, conventional microprocessors, application specific integrated circuits (ASICs), and field-programmable gate arrays (FPGAs).

Examples of microcontroller boards that are applicable in various embodiments include:
Raspberry Pi 4 Model B: a microcontroller with multiple I/O pins, capable of handling complex algorithms, sensor integration and display output, that supports a variety of programming languages.
Arduino Mega 2560 and Arduino Duo: popular for their simplicity, community support, and significant processing power for a hobbyist microcontroller board. The Arduino boards also have a large number of I/O pins and sufficient memory to handle a device with several sensors and control elements.
ESP32-WROOM-32: has built in wi-fi and Bluetooth capabilities, for the option of wireless transmission. The ESP3 offers a good balance between processing power and connectivity.
STM32 microcontrollers, particularly the STM32F407 and STM32L4 models, are recognized for their high performance and real-time operating system (RTOS) capabilities.
The MSP430FR6989 is an ultra-low-power microcontroller designed for applications where energy efficiency is paramount. This makes it a good fit for battery-operated medical devices that require long operational times without frequent recharging.

Other options include custom microcontroller solutions.

### Timer

Timers in microcontrollers are important for managing time-sensitive tasks, offering functions like precise delays, PWM generation, and system reliability. Examples of timers useful in various embodiments herein include:
16-bit Timer/Counter (TIMER1): Found in AVR microcontrollers like the ATmega328P, used for generating accurate time delays, PWM signals, and pulse durations.
8-bit Timer/Counter (TIMER0): A simpler timer in AVR microcontrollers, suitable for basic tasks like time delays and event counting.
32-bit Timer: Common in ARM Cortex-M microcontrollers (e.g., STM32), providing high resolution for advanced tasks like real-time clocks and high-frequency PWM.
Watchdog Timer (WDT): Ensures system reliability by resetting the microcontroller if it becomes unresponsive, critical in mission-critical applications.
PWM Timer: Found in microcontrollers like ESP32 and Arduino, used for controlling motors, LEDs, and other devices requiring variable signals.
Real-Time Clock (RTC): Tracks real-world time in applications like data logging, ensuring consistent timekeeping even when the main system is off.
Input Capture Timer: Captures the exact time of external events, useful in frequency measurement and pulse width analysis.
Output Compare Timer: Generates interrupts or toggles outputs at specific times, enabling precise periodic operations.

### Communications Interface/Wireless or Wired Transmitter

As used herein for purposes of the present disclosure, the term “wireless transmitter” is used to generally describe apparatus and systems relating to the wireless transmission of a signal. Any of a wide variety of wireless transmission devices and communications protocols may be employed in the various embodiments, including analog and digital transmission systems. Exemplary but non-limiting wireless transmitters that may be used include radio transmitters, cellular transmitters, LTE and LTE advanced systems, ZigBee™, Wi-Fi, and Bluetooth transmitters. Additionally, a plurality of wireless network and transmission systems may be employed without departing from the scope of the disclosure, including but not limited to wireless personal area networks, local area networks, mesh networks, metropolitan area and global area networks.

### Flow Limiting Device

The flow-limiting device subsystem is designed to regulate the flow of respiratory gases to prevent overventilation. The flow-limiting device can be a gradient-type or binary-type device, configured to impede the flow when the pressure or flow rate from the ventilation bag surpasses a maximum threshold value.

Gradient-type flow limiting devices are designed to gradually restrict or control the flow of gas or liquid based on a continuous range of variables such as pressure, flow rate, or other environmental conditions. These devices do not simply switch between on and off states but instead adjust the flow proportionally, allowing for more nuanced control of the flow rate. Examples include but are not limited to pressure regulators, variable orifice flow restrictors (U.S. Pat. No. 6,792,947B) or proportional valves.

Binary-type flow limiting devices operate in an all-or-nothing manner, meaning they switch between fully open (allowing flow) and fully closed (restricting flow) states. These devices are used when a simple on/off control of the flow is sufficient or desirable. Examples include but are not limited to ball valves, solenoid valves, automatic flow and pressure limiting valves (US20240350764A1) or check valves.

For example, this device may incorporate a deformable seal that fully blocks the flow path in response to excessive pressure or flow, ensuring the ventilation remains within safe parameters. This subsystem's primary function is to safeguard against excessive airflow. U.S. Pat. No. 12,017,008 discloses a pressure safety device for a bag valve mask discloses an automatic flow reduction valve useful as a flow limiting device in embodiments herein, and is incorporated herein by reference for all purposes.

According to an embodiment, the flow control valve as described in U.S. Pat. No. 6,792,947 B1 may be configured to automatically and variably limit the rate of gas flow from the bag to the mask between a predetermined minimum flow rate and a maximum flow rate, instead of fully blocking the flow path.

### User Interface

The assembly may include a user interface. Examples of a user interface include a touch sensitive display, or a simple LCD or LED display and optionally an input device or toggle such one or more physical or virtual pushbuttons.

Alternatively, the assembly can be adapted and integrated to be used with existing emergency setting monitors, for example, devices from Zoll, Stryker or Phillips.

The assembly can also be connected to an off-the-shelf screen, such as a raspberry pi LCD screen, or a microPC.

In further embodiments, a clock may be provided either separately or integrally with the processor to permit time stamping of data acquired during operation of the system disclosed herein.

In an embodiment, the user interface or display can be integrated into the sensor unit, e.g., element 4 in FIG. 1A or FIG. 1B.

### Alarm

The alarm subsystem operates based on the calculation of predicted maximum tidal volume or measured inspiratory flow time. The alarm subsystem may generate a notification (e.g. audible, visual, vibration, etc.) when the inspiratory time exceeds a predefined threshold value, alerting the user to potential overventilation.

The alarm system can include an audible alarm (e.g., buzzers or piezoelectric alarms that emit a sound when a threshold is exceeded, such as maximum tidal volume or pressure), or a visual alarm (e.g., LED indicators or small display screens that light up or change color to indicate an issue or an OLED displays (small, board-mounted displays that can show detailed alarm messages or ventilation data)).

### Software Flowchart

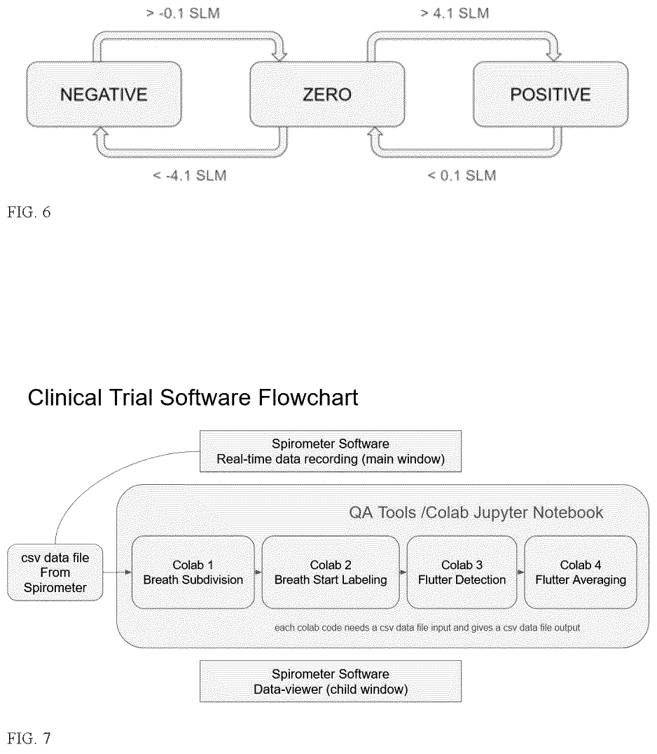

Certain embodiments provide a real-time method and system for detecting breaths and calculating inspiratory volume, inspiratory flow time in respiratory monitoring devices. The system utilizes pressure and flow rate measurement data to monitor and validate respiratory cycles, detect the start and end of a breath, and ensure breath integrity by verifying parameters such as inspiratory and expiratory volumes, peak pressure, and flow rates. As shown in FIG. 6, a breath detection algorithm leverages a state machine comprising three states: Positive, Zero, and Negative, transitioning based on flow rate thresholds. Breath validation criteria and leak recovery mechanisms ensure accuracy in respiratory assessments, while the official inspiratory volume is calculated using trapezoidal integration of flow data with readings updated every 10 milliseconds. In this system, incorporating a maximum tidal volume prediction, along with a flow-limiting valve mechanism, can significantly reduce dependence on high-end, expensive hardware, thereby making the entire monitoring process more cost-efficient.

In respiratory monitoring and ventilation devices, precise detection of breaths and accurate measurement of respiratory volumes are vital for patient safety and effective treatment. Additionally, achieving cost-efficiency with respiratory consumables is essential to meet the healthcare system's current operational requirements and avoid cross-contamination. Traditional systems often rely on cumbersome hardware and costly sensors. Embodiments disclosed herein enhance both the accuracy and reliability of breath detection and inspiratory volume calculation, whether or not precise real-time pressure and flow rate measurements are available by predicting maximum tidal volume based on inspiratory flow time, using a flow-limiting valve. Certain embodiments offer user feedback on optimal inspiratory flow time and total inspiratory time, employing a flow-limiting device to maintain safe tidal volumes and pressure levels.

### Definitions

Inspiratory Flow: The flow of air into the airway opening during the inspiratory phase of a breath. By convention, inspiratory flow is considered positive (above zero) in graphical representations.

Inspiratory Flow Time: The duration from the onset of inspiratory flow (when air begins to enter the airway opening) until the cessation of inspiratory flow. This period represents the active inhalation phase.

Inspiratory Time: The period from the beginning of inspiratory flow to the beginning of expiratory flow. Inspiratory time includes both the inspiratory flow time and any inspiratory pause time.

Inspiratory Volume: The total volume of air inhaled during the inspiratory phase of a breath, calculated by integrating the inspiratory flow over the inspiratory time.

Expiratory Volume: The total volume of air exhaled during the expiratory phase of a breath, often used to validate that a breath is complete by comparing it to inspiratory volume.

Should any leaks occur with the supraglottic device, the expiratory volume differing from the inspiratory volume would be a good indication of that.

Peak Pressure: The highest pressure measured during a breath cycle, typically occurring near the end of the inspiratory phase.

Peak Flow: The maximum flow rate achieved during a breath, usually recorded during the initial phase of inspiratory flow.

Embodiments herein include a method and system that utilize real-time flow rate and pressure data to detect breaths, validate respiratory cycles, measure inspiratory flow time and predict max inspiratory volumes accurately. In an embodiment, the system comprises a state machine with three states: Positive, Zero, and Negative. A breath is detected only when the system transitions sequentially through each state, thereby confirming a complete respiratory cycle. Breath validation is achieved by comparing inspiratory and expiratory volumes, pressure, and flow rates to predetermined thresholds. Depending on the capability of the hardware, other surrogates of pressure, flow, and inspiratory flow time will be utilized.

In an embodiment, with reference to FIG. 6, a breath detection algorithm operates based on a state machine with three states: Positive State, Zero State and Negative State. The Positive State indicates that the system has detected positive flow, transitioning from the Zero State when the flow rate exceeds a first threshold, e.g., 4.1 SLM. The system remains in this state until the flow rate drops below a second threshold, e.g., 0.1 SLM. The Zero State indicates no significant flow detected, typically the resting phase between breaths. The system enters this state from the Positive State if the flow rate falls below a threshold, e.g., 0.1 SLM, or from the Negative State if the flow rate exceeds a threshold, e.g., −0.1 SLM. If the system remains in the Zero State for more than one second with a pressure reading below a threshold, e.g., 5.5 cmH2O, a leak recovery (reset) process is initiated. The Negative State represents a phase of negative flow, signifying expiration. The system enters this state from the Zero State when the flow rate is below a threshold, e.g., −0.1 SLM and remains until the flow rate exceeds a threshold, e.g., −4.1 SLM. The transition from the Negative State back to Zero completes a respiratory cycle, marking the end of a breath.

Breath Validation Criteria: In an embodiment, a detected breath is validated if certain conditions are met, for example when one or more of the following conditions are met:
Inspiratory volume exceeds a threshold, e.g., 10 mL.
Expiratory volume is at least 50% of the inspiratory volume.
Peak pressure during the breath is greater than a threshold, e.g., 0.5 cmH2O.
Peak flow rate during the breath is greater than a threshold, e.g., 1 L/min.

Inspiratory Volume Calculation: In an embodiment, the inspiratory volume is determined by integrating the flow rate over the course of a complete breath. Integration may be performed using the trapezoidal rule, with volume accumulated as volume+=flow*time_difference, where time difference is the interval (e.g., 10-millisecond interval) between readings. Only positive flow values above 1 L/min are considered for integration, ensuring that minor fluctuations do not affect the volume calculation. The result may be returned as an integer.

According to an embodiment, a method for detecting and validating breaths in a respiratory monitoring system includes utilizing a state machine with Positive, Zero, and Negative states, detecting a complete breath upon sequential transitions through each state, and validating the detected breath based on predefined inspiratory and expiratory volume thresholds, peak pressure, peak flow rate, or a surrogate of pressure or flow.

According to an embodiment, a method for calculating inspiratory volume is provided, wherein flow rate data is integrated using the trapezoidal rule, updated at an interval, e.g., a 10-millisecond interval, and restricted to positive flow rates above a threshold flow rate, e.g., 1 L/min.

According to an embodiment, a system for respiratory monitoring performs real-time pressure and flow rate measurements, transitions through defined states, and includes a leak recovery mechanism triggered by extended Zero state duration and low pressure readings.

In an embodiment, with reference to FIG. 7, a clinical trial software module is provided to address imperfections in the data, wherein the software module may include various sub-modules as follows:

Breath subdivision module—This module investigates breaths for potential cases of two breaths that were merged together by the breath counting algorithm and lets the QA reviewer manually split breaths.

Breath Start Labeling module—The output from the clinical trial software defines an end time for each breath but does not define a start time. This module goes through every breath in a recording and has the reviewer manually label a start time. This is assisted by an automated detection which finds good defaults for each breath, to make labeling faster.

Flutter Detection module—The flow limiting valve can sometimes flutter during data collection. This happens most often when the correct conditions coincide with a flow limiting activation. This module automatically detects flutter and walks the QA reviewer through potential flutter detections, and then asks the reviewer to label start and end times for each instance.

Flutter Averaging module-Takes in a file that has been labeled via the flutter detection module and then applies a moving average filter to regions within flutter labels. This is automated and the output file is available for download almost instantly, no manual oversight needed.

It is intended that the sub-modules will be run in order, each using the output of the previous step. If a step is not desired, it can be skipped.

### User Interface

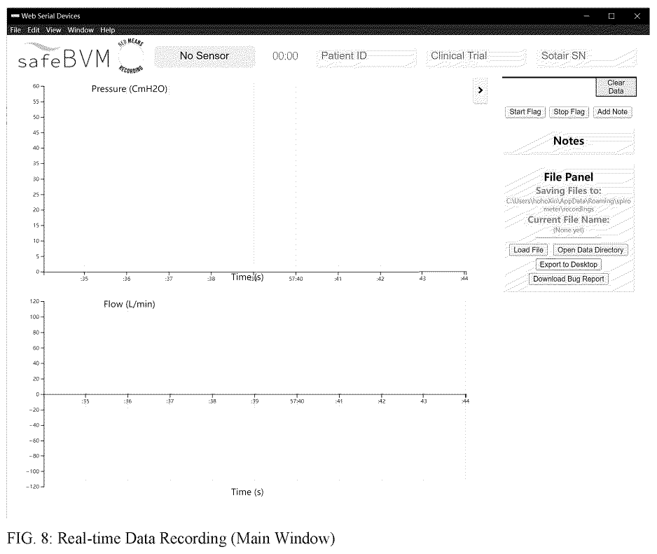

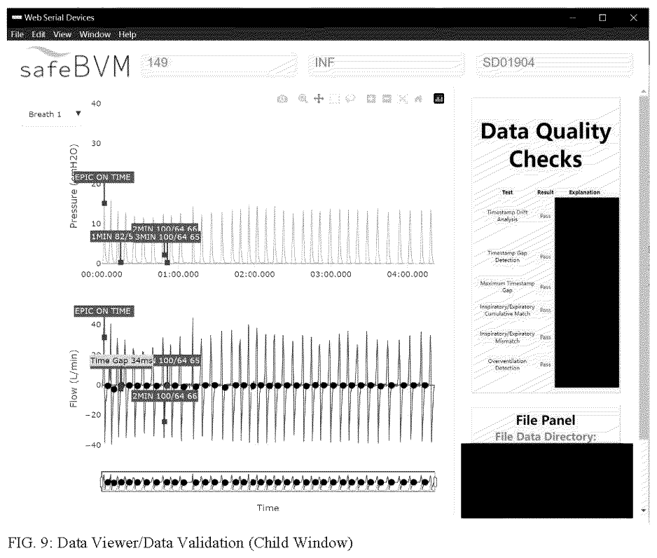

FIG. 8 shows an example of a real-time data recording displayed on a main window on a user interface. FIG. 9 shows an example of a data viewer and data validation window displayed on the user interface.

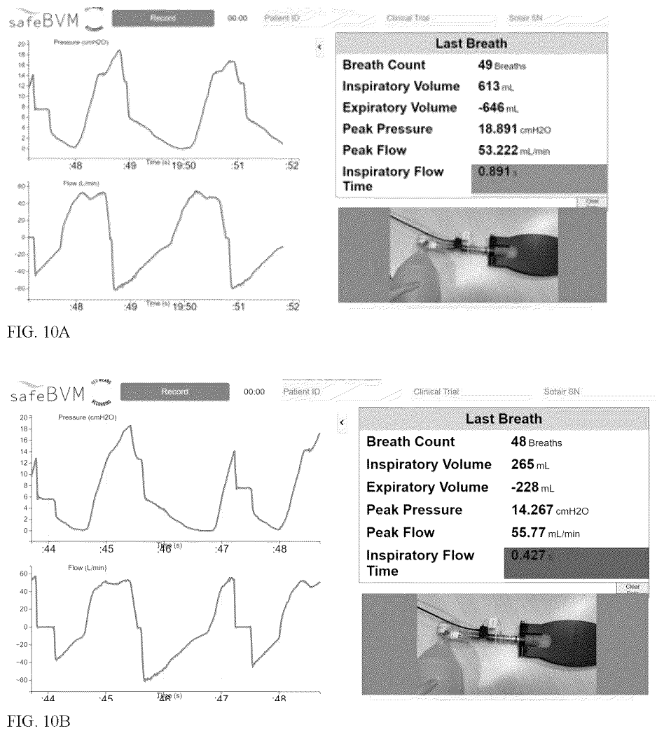

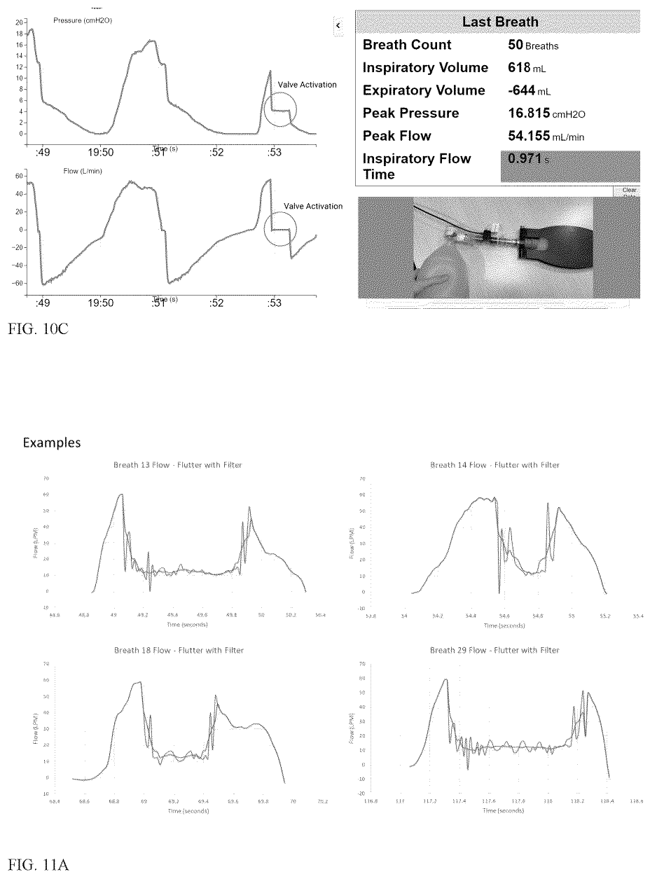

FIG. 10A, FIG. 10B and FIG. 10C show examples displays of Inspiratory Flow time Feedback & Flow Limiting Valve Activation according to embodiments. As seen in FIG. 10B, the inspiratory flow time has been flagged red as the value (0.427 s) is outside of the threshold range of 0.8-1.1 seconds. Similarly, in FIG. 10A the inspiratory flow time has been flagged green when the value (0.891 s) was within the range for positive reinforcement. As shown, breaths delivered with flow limiting valve that are in the “green” inspiratory flow time range have a more optimum flow tidal volume in line with recommendations, while those in red have higher probability of being unsafe tidal volumes for the patient. Here, the optimum range for inspiratory flow time will depend on the flow limiting threshold. For example, with a flow limiting threshold of 45-55 LPM, an inspiratory flow time of 0.8-1.1 will be adequate for ˜600-650 ml max tidal volume. If the flow limiting threshold is lower, then a higher inspiratory flow time range would be required. In an embodiment, inspiratory flow time's range based on different combinations can range between 0.3-2 seconds. These ranges are linked to the theoretical max values, however the range for standard resuscitator bags will lie between 0.7-1.5 seconds. Similarly, as shown in FIG. 10C, valve activation has been flagged based on patterns. The user and the trainer can be provided with and receive feedback by detecting these patterns.

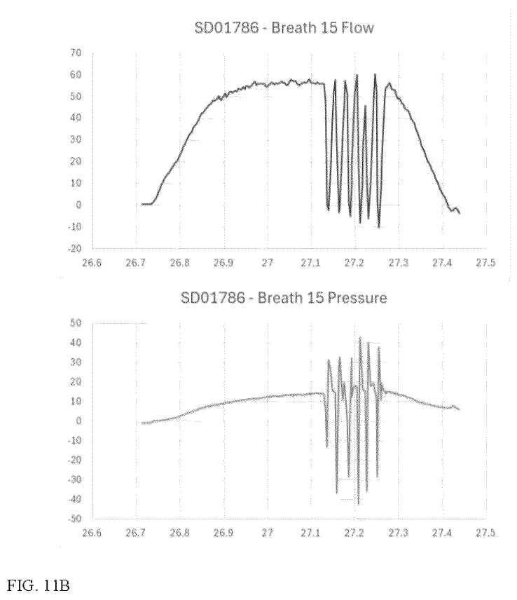

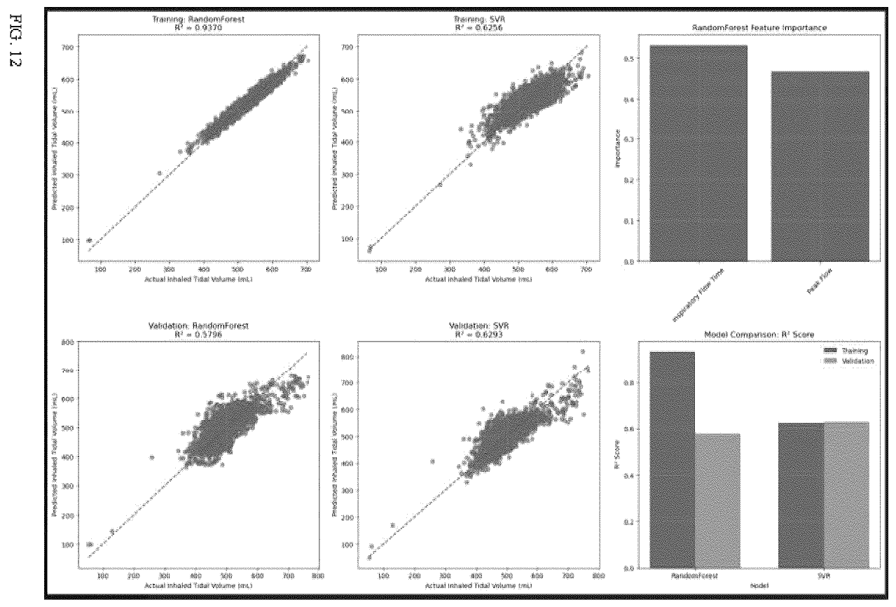

FIG. 11A and FIG. 11B show examples of flutter characterized by an aberrant waveform due to activation of the flow limiting valve and the readings being picked up by the sensor. These readings are a physical phenomenon that result in turbulent airflow affecting the accuracy of the sensors. Depending on the type of the flow limiting valve, these waveforms will be unique and will impact the pink curve and the max tidal volume predictions. Use of a smoothening function advantageously enables improvement of the accuracy of the maximum tidal volume prediction

### EXAMPLES

The embodiments herein advantageously make it possible to maintain a certain adjustable pressure in the manual bag in the manual ventilation mode. This improves the tactile properties of the manual bag since the operator can adjust the pressure such that the operator has a convenient palpable contact with the lungs of the patient via the manual bag.

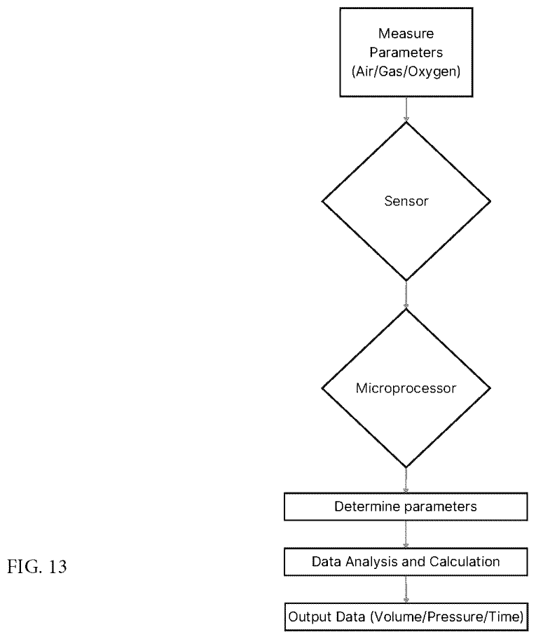

FIG. 13 shows an example process flow according to an embodiment. Upon compression of the bag or balloon, the sensor measures various parameters of the passage of air/oxygen/gas inside the flow path and provides the data to a microcontroller which determines various parameters, such as determining or calculating volumes, velocities pressures of the insufflations, and the data may be analyzed (e.g., to calculate other parameters as discussed herein) and the data and parameters may be displayed.

In one example embodiment, the flow measurement sensor mechanism or flowmeter in the device, which monitors/measures the volume and pressure of air, oxygen, or gas during each insufflation, can be a propeller, turbine, or anemometer system. The system is activated by the manual compression of the bag, with the resulting data collected and analyzed by a connected microprocessor (e.g., controller). The device can include an integrated or additional manometer to detect and monitor the pressure of the gas produced by the bag's compression, ensuring precise and reliable measurements.

In another example embodiment, the flowmeter with which the device measures the parameters of the passage of the air/oxygen/gas inside it, deducing the volumes, the speeds (and potentially, for indirect measurement, the pressures) of the insufflations can be a “hot wire flowmeter”. The data is then transmitted to a microprocessor, where it is analyzed and displayed.

In yet another example embodiment, the flowmeter with which the device measures the parameters of the passage of air/oxygen/gas inside it, deducing the volumes, velocities and pressures of the insufflations, may include a “Pitot tube”. The data is then transmitted to a microprocessor, where it is analyzed and displayed.

In yet another example embodiment, the flowmeter with which the device measures the parameters of the air/oxygen/gas passage inside it, deducing the volumes and pressures of the insufflations, may include a “Venturi tube”. The data is then transmitted to a microprocessor, where it is analyzed and displayed.

In yet another example embodiment, the flowmeter with which the device measures the parameters of the air/oxygen/gas passage inside it, deducing the pressures and volumes of the insufflations, may include a “differential pressure gauge” or “calibrated orifice” or “diaphragm”. The data is then transmitted to a microprocessor, where it is analyzed and displayed.

### Machine Learning-Based Tidal Volume Estimation

According to an embodiment, an approach is provided to turn the two breath-level signals discussed above—peak inspiratory flow, inspiratory flow time, or both together—into a reliable, numeric estimate of tidal volume that works in real time. To do so, in an embodiment each inspiration is segmented, and paired every breath's peak flow (PF) and inspiratory time (TI) with the ground-truth tidal volume obtained by numerical integration of the flow trace. On this breath-by-breath table, two contrasting regression models were trained using a small data set: a Random Forest, which stitches together hundreds of local decision rules, and a Support Vector Regressor (SVR) with an RBF kernel, which fits a single smooth surface across the whole data set. The Random-Forest showed an almost planar rise that steepens once peak flow exceeds about 60 L min⁻¹; fitting a simple plane through those outputs yields VT≈3.92×PF×TI+307 mL and an in-sample R² of 0.71. The SVR was noticeably smoother, reflecting the algorithm's bias toward a single global function, and can be summarized as VT≈4.08×PF×TI+288 mL with an R² of 0.52 on the same provider.

To test whether either model would survive a change of operator, the weights learned from one provider were frozen and applied to 1918 breaths from a new provider. The Random Forest, which had achieved an almost perfect R² of 0.94 during training, dropped to 0.58 on the new provider—strong evidence of over-fitting. The SVR, on the other hand, held essentially the same explanatory power across both datasets, scoring an R² of 0.63 with a slightly lower validation error (RMSE 38.4 mL versus 40.9 mL). FIG. 12 confirms that inspiratory time and peak flow contribute in nearly equal measure—about 53 percent and 47 percent, respectively—to the final estimate, a balance that remains stable across algorithms.

In short, the Random-Forest model gives a vivid picture of how a single clinician's bagging behavior translates into delivered volume, but its piece-wise nature makes it sensitive to individual style. The SVR sacrifices some in-sample accuracy to gain robustness; it delivers a portable, device-friendly equation that predicts tidal volume within about ±40 mL even when the operator changes. Taken together, FIG. 12 and Tables 1 and 2, below, document the full journey: from raw waveform to model training, through visual exploration of each predictive surface, to a head-to-head validation that identifies SVR as the safer choice for deployment.

TABLE 1
From Raw Waveform to Model Inputs

| Stage | Action | Result |
|---|---|---|
| A. Breath | Detect inspiration start (t0) and | Positions t0 and t1 |
| segmentation | end (t1) from zero-flow crossings | for each breath |
| | plus a small hysteresis band. | |
| B. Feature | Peak Flow (PF) = max flow | Feature vector x = |
| extraction | between t0 and t1 (L/min) − | [PF, TI] |
| | Inspiratory Flow Time (TI) = | |
| | t1 − t0 (s) | |
| C. Ground- | Integrate the same flow samples | Inhaled tidal |
| truth label | | volume in mL |
| (training only) | | |

TABLE 2
Two exemplary models:

| Model | Core Idea | Why It Helps |
|---|---|---|
| Random- | Average of ~200 decision | Captures sharp non-linear |
| Forest | trees, each splitting the | effects; shows feature |
| Regressor | PF-TI space differently. | importance (TI ≈ 53%, |
| | | PF ≈ 47%). |
| Support- | Maps inputs into a high- | Generalizes well, resists |
| Vector | dimensional space and fits | overfitting. |
| Regressor | the flattest surface that | |
| (SVR) | encloses most points. | |

Both models predict: VT_pred=f(PF, TI)

SVR selected for production due to stability across datasets.

Real-Time Deployment Pipeline
During each inspiration:
   Detect t0/t1 (start and end of breath);
   Compute PF and TI (<0.1 ms);
Predict VT:
   Input [PF, TI] to SVR model;
   Execution time≈150 μs (Cortex-M4);
Output:
   Display VT_pred for clinician use;
   Log alongside integrated flow volume for analytics and alarms.

The present embodiments advantageously improve upon existing system by adding Inspiratory Time as a second dimension and using a learned, nonlinear model instead of fixed ratio estimation.

It should be noted that other machine learning algorithms, or any algorithm type (e.g., supervised, semi-supervised, unsupervised and reinforcement) may be used as would be apparent to one skilled in the art.

### Clinical Feedback and Data Logging

The system may include a visual feedback interface that displays:
Real-time waveforms (flow, volume, pressure).
Alerts for abnormal ventilation.
Summary reports for quality review.

Data is logged locally or uploaded to software, e.g., SotairIQ for further analysis.

### Integrated Training Platform

In certain embodiments, a training solution is provided that aims to train practitioners in flow for manual ventilation. Most practitioners have never had real-time feedback on their manual ventilation performance measuring critical characteristics of volume, pressure and flow rate. The training solution provides waveform visualization with feedback, builds muscle memory and flow proficiency through coaching and gamified training, enabling clinical confidence.

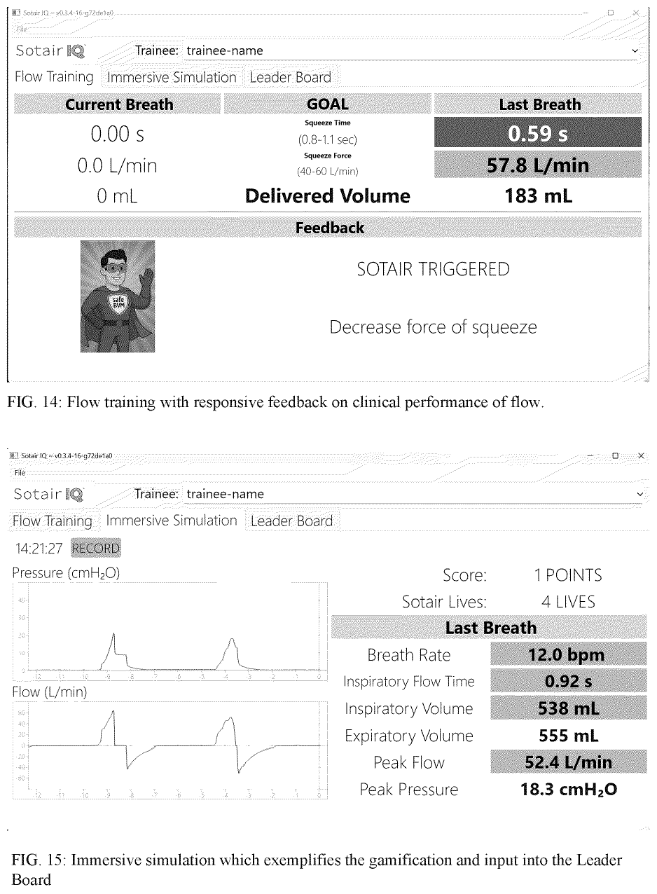

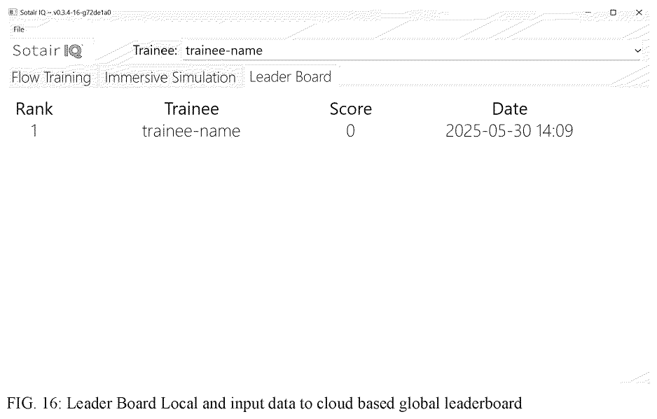

This training solution transforms an interactive gamified environment into a skills-based learning experience designed to enhance engagement, retention, and performance by applying game mechanics to technical training content. Game elements (including competitive challenges, points, levels, leaderboards, feedback, etc.) into real-world training environments to motivate learners, simulate realistic scenarios and reinforce knowledge through repetition and reward. FIG. 14, FIG. 15 and FIG. 16 illustrate examples of user interface displays embodying the interactive gamified environment according to an embodiment. For example, in certain embodiments, the system may include a training module featuring:
Scenario-based simulation.
Gamified mechanics: points, levels, achievements, leaderboards.
Adaptive difficulty and personalized progression.
Waveform-based feedback.
Analytics dashboard for instructors.

This enhances skill retention and builds clinician confidence under stress. Overall goals are to:
shift the standard of care in manual ventilation from pressure to flow in manual ventilation.
convert passive learning into active participation
improve knowledge retention and decision-making
drive self—directed learning through immediate feedback and goal setting
building that emotional resilience, control and grit through simulation of real-world stressors.

Additional advantages of the various embodiments include:
1. Proximal Sensing: Reduces error by sensing close to patient.
2. Machine Learning Integration: Enables accurate VT prediction using simple, low-cost inputs.
3. Real-Time Feedback: Supports better clinical decisions.
4. Training Mode: Builds proficiency with interactive coaching.
5. System Versatility: Supports standalone, embedded, and cloud-connected operation.

U.S. Pat. No. 12,017,008 (Pressure safety device for bag valve mask) discloses bag valve mask systems, device components, and methods useful in embodiments herein, and is incorporated herein by reference for all purposes. The following patents and patent applications provide additional context, features, devices, sensors, device components and methods useful in various embodiments herein, and each is incorporated herein by reference: U.S. Pat. No. 11,596,753B2, US20220023558A1, US20080053445, US20060060199, U.S. Pat. No. 11,712,531B2, U.S. Pat. No. 11,135,383B2, U.S. Pat. Nos. 11,129,950, 10,974,002, WO2019077493A1, WO2018065448A1, WO2002015968, and WO2013140087A1.

Integrated IoT Module for On Sensor Analysis and Secure Web Application Delivery

In one embodiment, the integrated manual ventilation monitoring system includes a sensor module and an integrated IoT type processing unit (e.g., processor) directly coupled thereto. In this embodiment, raw sensor data is acquired, measured, and analyzed locally on the processing unit—eliminating reliance on host machine USB ports, which are often blocked by firewalls or security patches. In a further embodiment, the locally computed breath metrics (i.e., final breath readings) are streamed via a secure, webcast like system to a browser based web application. Authorized users may access this web application through a standard web browser using a unique username and password, thereby linking each data stream to its intended recipient. This browser based architecture obviates the need for customer side software downloads or installations, addressing common restrictions on downloads and software installation on customer laptops. According to another embodiment, the IoT processing unit incorporates a machine learning algorithm for real time analysis and is powered via a standard adapter. Together, these embodiments enable secure, rapid deployment—even in environments where Wi Fi or Internet access is limited or prohibited—by processing data locally and streaming only preprocessed breath readings to the web application.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosed subject matter (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or example language (e.g., “such as”) provided herein, is intended merely to better illuminate the disclosed subject matter and does not pose a limitation on the scope of the invention unless otherwise claimed. No language in the specification should be construed as indicating any non-claimed element as essential to the practice of the invention.

Certain embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A system for reducing the risk of overventilating a subject in need of ventilation, the system comprising:
a ventilation assembly comprising a ventilation bag, a patient interface and an automatic flow reduction valve configured to impede flow to a subject wearing the ventilation bag when a pressure or flow rate in a flow path from the bag to the subject exceeds a maximum threshold value, the assembly further comprising a ventilation sensor positioned in or near to the flow path and configured to measure the pressure or flow rate in the flow path, a timer, and a user interface, the ventilation assembly configured to communicate via the user interface at least one signal related to a maximum tidal volume determined based on signals from the ventilation sensor.

2. The system of claim 1, wherein the patient interface comprises a face mask or a laryngeal mask for supraglottal ventilation or an endotracheal tube.

3. The system of claim 1, wherein the at least one signal related to the maximal tidal volume is determined based on one or more of an inspiratory time, an inspiratory flow time, and a peak flow rate.

4. The system of claim 3, wherein the ventilation sensor detects a start and an end of an inspiratory pulse and the inspiratory flow time is measured based on the time difference between the start and the end of the inspiratory pulse.

5. The system of claim 1, wherein the ventilation sensor includes one of a pressure sensor or a flow sensor located along the flow path within a device housing the automatic flow reduction valve.

6. The system of claim 3, wherein the start of the inspiratory pulse is triggered by a pressure or flow rate or both above a start threshold value and the end of the inspiratory pulse is triggered by a subsequent pressure or flow reading below an end threshold value.

7. The system of claim 1, wherein the flow reduction valve comprises a deformable seal having an open position and a closed position wherein the deformable seal is configured to deform to fully block the flow path in response to the pressure or flow rate exceeding the maximum threshold value.

8. The system of claim 1, wherein the at least one signal related to a maximal tidal volume is a warning signal triggered when an inspiratory flow time exceeds an inspiratory flow time threshold value.

9. The system of claim 8, wherein the inspiratory flow time threshold value is a maximum tidal volume determined based on an inspiratory time and flow characteristics of the automatic flow reduction valve.

10. The system of claim 1, wherein the system further includes a processor configured to implement machine learning to determine the maximal tidal volume.

11. An assembly comprising an automatic flow reduction valve with an integrated ventilation sensor and a processor, wherein the automatic flow reduction valve is configured to be mated between a ventilation bag and a patient interface and is configured to impede flow of air in a flow path from the bag to a subject in need of ventilation wearing the patient interface when the bag is compressed by a user causing a pressure or flow rate from the bag to the subject via the patient interface to exceed a maximum threshold value, and wherein the ventilation sensor is configured to detect the pressure or the flow rate of air in the flow path, and the processor is configured to measure an inspiratory flow time based on the detected pressure or flow rate at multiple time points in a ventilation cycle and to use the inspiratory flow time to generate a signal used to notify the user of a potential overventilation condition.

12. The assembly of claim 11, wherein the flow reduction valve comprises a deformable seal having an open position and a closed position wherein the deformable seal is configured to deform to fully block the flow path in response to the pressure or flow rate exceeding the maximum threshold value.

13. The assembly of claim 11, wherein the ventilation sensor includes one of a pressure sensor or a flow sensor located along the flow path within a device housing the automatic flow reduction valve.

14. A method for avoiding overventilation of a subject in need of ventilation, the method comprising:
(a) providing a ventilation assembly comprising a ventilation bag, a patient interface and an automatic flow reduction valve configured to impede flow to the patient interface when a pressure or flow rate in a flow path from the bag exceeds a maximum threshold value, the assembly further comprising a ventilation sensor positioned in or near to the flow path and configured to measure the pressure or flow rate in the flow path and a timer, the ventilation assembly configured to display at least one signal related to a maximum tidal volume determined based on signals from the ventilation sensor;
(b) positioning the patient interface on a subject in need of ventilation; and
(c) compressing the ventilation bag to deliver breathing gas to the subject and using the at least one signal related to the maximal tidal volume to avoid the overventilation of the subject.

15. The method of claim 14, wherein the automatic flow reduction valve comprises a deformable seal having an open position and a closed position wherein the deformable seal is configured to deform to fully block the flow path in response to the pressure or flow rate exceeding the maximum threshold value.

16. The method of claim 15, wherein the at least one signal related to the maximal tidal volume is based on one or more of an inspiratory time, an inspiratory flow time, and a peak flow rate.

17. The method of claim 16, wherein the at least one signal related to a maximal tidal volume is a warning signal triggered when an inspiratory flow time exceeds an inspiratory flow time threshold value.

18. The method of claim 14, wherein machine learning is used to determine the maximal tidal volume.

19. The method of claim 14, wherein the ventilation sensor includes one of a pressure sensor or a flow sensor located along the flow path within a device housing the automatic flow reduction valve.
